(19) United States
(12) Patent Application Publication
(10) Pub. No.: US 2022/0031227 A1
(43) Pub. Date: Feb. 3, 2022

(54) DEVICE AND METHOD FOR DIAGNOSING GASTRIC LESION THROUGH DEEP LEARNING OF GASTROENDOSCOPIC IMAGES

(71) Applicant: INDUSTRY ACADEMIC COOPERATION FOUNDATION, HALLYM UNIVERSITY, Chuncheon-si (KR)

(72) Inventors: Bum-Joo CHO, Seoul (KR); Chang Seok BANG, Chuncheon-si (KR); Se Woo PARK, Hwaseong-si (KR); Jae-Jun LEE, Chuncheon-si (KR); Jae-Ho CHOI, Chuncheon-si (KR); Seok-Hwan HONG, Seoul (KR); Yong-Tak YOO, Seoul (KR)

(21) Appl. No.: 17/278,962

(22) PCT Filed: Sep. 25, 2019
(86) PCT No.: PCT/KR2019/012448

§ 371 Date: Mar. 23, 2021

(30) Foreign Application Priority Data
Oct. 2, 2018 (KR) 10-2018-0117823

(51) Int. Cl. A61B 5/00; G06T 7/00; G06K 9/62; A61B 1/273; G06N 3/04; G16H 30/40; G16H 50/20

(52) U.S. Cl. CPC A61B 5/4216; G06T 7/0012; G06K 9/6262; A61B 5/7267; A61B 1/2736; G06N 3/0454; G06T 2207/20081; G16H 50/20; G06T 2207/10068; G06T 2207/20084; G06T 2207/30092; G06T 2207/30096; G16H 30/40

(57) ABSTRACT

A method for diagnosing a gastric lesion from endoscopic images is provided. The method comprises: acquiring a plurality of gastric lesion images; generating a dataset by linking the plurality of gastric lesion images with patient information; preprocessing the dataset in a way that is applicable to a deep learning algorithm; and building an artificial neural network by training the artificial neural network by using the preprocessed dataset as input and gastric lesion classification results as output.

### CROSS-REFERENCE TO RELATED APPLICATIONS

This application claims the benefit under 35 U.S.C. section 371, of PCT International Application No.: PCT/KR2019/012448, filed on Sep. 25, 2019, which claims foreign priority to Korean Patent Application No.: 10-2018-0117823, filed on Oct. 2, 2018, in the Korean Intellectual Property Office, both of which are hereby incorporated by reference in their entireties.

### BACKGROUND OF THE DISCLOSURE

### Field of the Disclosure

The present disclosure relates to a device and method for diagnosing a gastric lesion through deep learning of gastroendoscopic images.

### Related Art

Cells, the smallest units that make up the human body, divide by intracellular regulatory functions when normal, and maintain cell balance while growing, dying, and disappearing. When a cell is damaged for some reason, it is treated and regenerated to serve as a normal cell, but if it does not recover, it will die by itself. However, cancer is defined as a condition in which abnormal cells that do not control proliferation and inhibition for many reasons are not only excessively proliferating but also invade surrounding tissues and organs, resulting in mass formation and normal tissue destruction. Cancer is a cell proliferation that cannot be inhibited, and it destroys normal cell and organ structure and function, so its diagnosis and treatment are very important.

Cancer is a group of diseases in which cells proliferate infinitely and interfere with normal cellular functions, the most common examples of which are lung cancer, gastric cancer (GC), breast cancer (BRC), and colorectal cancer (CRC) though they can develop in virtually any tissue. In the past, cancer was diagnosed based on external changes in biological tissue caused by growth of cancer cells, but recently, diagnosis and detection of cancer using a trace amount of biomolecules existing in biological tissues or cells such as blood, glycol chain, DNA, or the like, have been attempted. However, the most generally used method for diagnosis of cancer is via usage of a tissue sample acquired by a biopsy or usage of an image.

Globally, gastric cancer is more prevalent in South Korea and Japan, whereas the incidence rates of gastric cancer are rather low in Western countries such as the United States and Europe. In South Korea, gastric cancer is the cancer of highest incidence and also the second leading cause of cancer death after lung cancer. As for types of gastric cancer, 95% of gastric cancers are adenocarcinomas, which originate in glandular cells of the mucosa that lines the stomach. Other cancers include lymphoma, which originates in the lymphatic system, and gastrointestinal stromal tumor, which originates in stromal tissues.

Among these, the biopsy is disadvantageous in that it causes great pain to the patient and is not only expensive but also takes a long time to diagnose. In addition, if a patient actually has cancer, cancer metastasis may be induced during the biopsy process. For a region from which a tissue sample cannot be taken by a biopsy, it is not possible to make a disease diagnosis unless a suspicious lesion is surgically removed.

In diagnosis using images, cancer is identified based on X-ray images, nuclear magnetic resonance (NMR) images acquired using a contrast agent to which a disease target substance is attached, and the like. However, there is a drawback that such diagnosis based on images may cause misdiagnosis depending on the skill level of a clinician or an interpreting physician, and greatly depends on the accuracy of the device that acquires the images. Furthermore, even the most accurate devices cannot detect tumors as small as or smaller than several mm, which makes it difficult to detect cancer in the initial stages. Also, in order to obtain a picture, the patient or disease holder is exposed to high-energy electromagnetic waves that can induce gene mutations, which may cause other diseases as well, and another drawback is that the number of diagnoses made through imaging is limited.

Most early gastric cancers (ECG) cause no clinical symptoms or signs, which make it difficult to detect and treat them at the right time without a screening strategy. Moreover, patients with premalignant lesions such as dysplasia are at high risk of gastric cancer.

In the past, neoplasms of the stomach were identified as cancers primarily based on their shapes and sizes in the stomach as observed on gastroendoscopic images and then confirmed as cancer by a biopsy. This method, however, will produce different diagnoses depending on the doctor's experience and does not ensure accurate diagnosis in areas where there are no doctors.

Moreover, the detection of abnormal lesions is usually based on abnormal morphology or color changes in the mucosa, and it is known that diagnostic accuracy is improved through learning and optical techniques or chromoendoscopy. The application of endoscopic imaging technologies such as narrow-band imaging, confocal imaging or magnifying techniques (so-called image-enhanced endoscopy) is also known to enhance diagnostic accuracy.

However, examination solely with white-light endoscopy remains the most routine form of screening, and standardization of the procedure and improvements in the interpretation process to resolve the interobserver and intraobserver variability are needed in image-enhanced endoscopy.

The related art to the present disclosure is disclosed in Korean Unexamined Patent Publication No. 10-2018-0053957.

### SUMMARY

The present disclosure has been made in an effort to solve the aforementioned problems occurring in the related art and to provide a gastric lesion diagnostic device which can diagnose a gastric lesion by collecting white-light gastroendoscopic images acquired by an endoscopic video imaging device and feeding them into a deep learning algorithm.

The present disclosure has been made in an effort to solve the aforementioned problems occurring in the related art and to provide a gastric lesion diagnostic device which provides a deep learning model for automatically classifying gastric tumors based on gastroendoscopic images.

The present disclosure has been made in an effort to solve the aforementioned problems occurring in the related art and to provide a gastric lesion diagnostic device which can diagnose a barely noticeable gastric tumor by evaluating multiple image data in real time which is acquired when a doctor (user) examines the gastric tumor using an endoscopic device.

The present disclosure has been made in an effort to solve the aforementioned problems occurring in the related art and to provide a gastric lesion diagnostic device which can diagnose and predict gastric cancer or gastric dysplasia by automatically classifying a neoplasm of the stomach based on gastroendoscopic images acquired in real time.

However, the technical problems to be solved in the present disclosure are not limited to the above-described ones, and other technical problems may be present.

As a technical means for solving the above-mentioned technical problems, an exemplary embodiment of the present disclosure provides a method for diagnosing a gastric lesion from endoscopic images, the method including: acquiring a plurality of gastric lesion images; generating a dataset by linking the plurality of gastric lesion images with patient information; preprocessing the dataset in a way that is applicable to a deep learning algorithm; and building an artificial neural network by training the artificial neural network by using the preprocessed dataset as input and gastric lesion classification results as output.

According to an exemplary embodiment of the present disclosure, the method may further include performing a gastric lesion diagnosis through the artificial neural network after passing a new dataset through the preprocessing process.

According to an exemplary embodiment of the present disclosure, the generating of a dataset may include classifying the dataset as a training dataset required for training the artificial neural network or a validation dataset for validating information on the progress of the training of the artificial neural network.

According to an exemplary embodiment of the present disclosure, the validation dataset may be a dataset that is not redundant with the training dataset.

According to an exemplary embodiment of the present disclosure, the validation dataset may be used for evaluating the performance of the artificial neural network when a new dataset is fed as input into the artificial neural network after passing through the preprocessing process.

According to an exemplary embodiment of the present disclosure, the acquisition of images may include receiving gastric lesion images acquired by an imaging device with which an endoscopic device is equipped.

According to an exemplary embodiment of the present disclosure, the preprocessing may include: cropping a peripheral area of a gastric lesion image included in the dataset around the gastric lesion to a size applicable for the deep learning algorithm in such a way that the gastric lesion is not included in the image; shifting the gastric lesion image in parallel upward, downward, to the left, or to the right; rotating the gastric lesion image; flipping the gastric lesion image; and adjusting colors in the gastric lesion image, wherein the gastric lesion image may be preprocessed in a way that is applicable to the deep learning algorithm by performing at least one of the preprocessing phases.

According to an exemplary embodiment of the present disclosure, the preprocessing may include augmenting image data to increase the amount of gastric lesion image data, wherein the augmenting of image data may include augmenting the gastric lesion image data by applying at least one of the following: rotating, flipping, cropping, and adding noise into the gastric lesion image data.

According to an exemplary embodiment of the present disclosure, the building of a training model may include building a training model in which a convolutional neural network and a fully-connected neural network are trained by using the preprocessed dataset as input and the gastric lesion classification results as output.

According to an exemplary embodiment of the present disclosure, the preprocessed dataset may be fed as input into the convolutional neural network, and the output of the convolutional neural network and the patient information may be fed as input into the fully-connected neural network.

According to an exemplary embodiment of the present disclosure, the convolutional neural network may produce a plurality of feature patterns from the plurality of gastric lesion images, wherein the plurality of feature patterns may be finally classified by the fully-connected neural network.

According to an exemplary embodiment of the present disclosure, the building of an artificial neural network may include performing training by applying training data to a deep learning algorithm architecture including a convolutional neural network and a fully-connected neural network, calculating the error between the output derived from the training data and the actual output, and giving feedback on the outputs through a backpropagation algorithm to gradually change the weights of the artificial neural network architecture by an amount corresponding to the error.

According to an exemplary embodiment of the present disclosure, the performing of a gastric lesion diagnosis may include classifying the gastric lesion diagnosis as at least one of the following categories: advanced gastric cancer, early gastric cancer, high-grade dysplasia, and low-grade dysplasia.

An exemplary embodiment of the present disclosure provides a device for diagnosing a gastric lesion from endoscopic images, the device including: an image acquisition part for acquiring a plurality of gastric lesion images; a data generation part for generating a dataset by linking the plurality of gastric lesion images with patient information; a data preprocessing part for preprocessing the dataset in a way that is applicable to a deep learning algorithm; and a training part for building an artificial neural network by training the artificial neural network by using the preprocessed dataset as input and gastric lesion classification results as output.

According to an exemplary embodiment of the present disclosure, the device may further include a lesion diagnostic device for performing a gastric lesion diagnosis through the artificial neural network after passing a new dataset through the preprocessing process.

The above-mentioned solutions are merely exemplary and should not be construed as limiting the present disclosure. In addition to the above-described exemplary embodiments, additional embodiments may exist in the drawings and detailed description of the disclosure.

According to the above-described means for solving the problems of the present disclosure, it is possible to diagnose a gastric lesion by collecting white-light gastroendoscopic images acquired with an endoscopic video imaging device and feeding them into a deep learning algorithm.

According to the above-described means for solving the problems of the present disclosure, it is possible to provide a deep learning model for automatically classifying gastric tumors based on gastroendoscopic images.

According to the above-described means for solving the problems of the present disclosure, it is possible to diagnose a barely noticeable gastric tumor by learning multiple image data in real time which is acquired when a doctor (user) examines the gastric tumor using an endoscopic device.

According to the above-described means for solving the problems of the present disclosure, it is possible to significantly reduce cost and labor, compared with the existing gastroendoscopy which requires a doctor's experience, by learning images acquired with an endoscopic video imaging device and classifying gastric lesions.

According to the above-described means for solving the problems of the present disclosure, it is possible to obtain objective and consistent interpretation and reduce potential mistakes and misinterpretation by an interpreting doctor, since a gastric lesion can be diagnosed and predicted with the above gastric lesion diagnostic device based on gastroendoscopic images acquired with an endoscopic video imaging device, and it is also possible to use the above gastric lesion diagnostic device as an aid for clinical decision.

However, advantageous effects to be achieved in the present disclosure are not limited to the above-described ones, and other advantageous effects may be present.

### BRIEF DESCRIPTION OF THE DRAWINGS

FIG. 1 is a schematic diagram of a lesion diagnostic device according to an exemplary embodiment of the present disclosure.

FIG. 2 is a schematic block diagram of a lesion diagnostic device according to an exemplary embodiment of the present disclosure.

FIG. 3 is a view illustrating an example of building an artificial neural network in a lesion diagnostic device according to an exemplary embodiment of the present disclosure.

FIG. 4 is an operation flow chart of a method for diagnosing a gastric lesion from endoscopic images according to an exemplary embodiment of the present disclosure.

### DESCRIPTION OF EXEMPLARY EMBODIMENTS

Hereinafter, embodiments of the present disclosure will be described in detail with reference to the accompanying drawings so that those skilled in the art can easily carry out the present disclosure. It should be understood, however, that the present disclosure may be embodied in many different forms and should not be construed as limited to the embodiments set forth herein. In the drawings, the same reference numbers are used throughout the specification to refer to the same or like parts.

Throughout this specification, it will be understood that, when a certain portion is referred to as being “connected” to another portion, this means not only that the certain portion is “directly connected” to the another portion, but also that the certain portion is “electrically connected” or “indirectly connected” to the another portion with an intervening element therebetween.

Throughout this specification, it will be understood that, when a certain member is located “on”, “above”, “on the top of”, “under”, “below”, or “on the bottom of” another member, this means not only that the certain member comes into contact with the another member, but also that there is an intervening member between the two members.

Throughout this specification, it will be understood that, when a certain portion “includes” a certain element, this does not preclude the presence of another element but the certain portion may include another element unless the context clearly dictates otherwise.

The present disclosure relates to a gastric lesion diagnostic device and method including a deep learning model for classifying gastric tumors based on gastroendoscopic images acquired from an endoscopic device and evaluating the performance of the device. The present disclosure allows for automatically diagnosing a neoplasm of the stomach by interpreting gastroendoscopic images based on a convolutional neural network.

The present disclosure enables the diagnosis and prediction of gastric cancer or gastric dysplasia by computer-training a convolutional neural network, which is a type of deep learning algorithm, on a dataset of gastroendoscopic picture images, interpreting newly input gastroendoscopic pictures, and therefore automatically classifying a neoplasm of the stomach in the pictures.

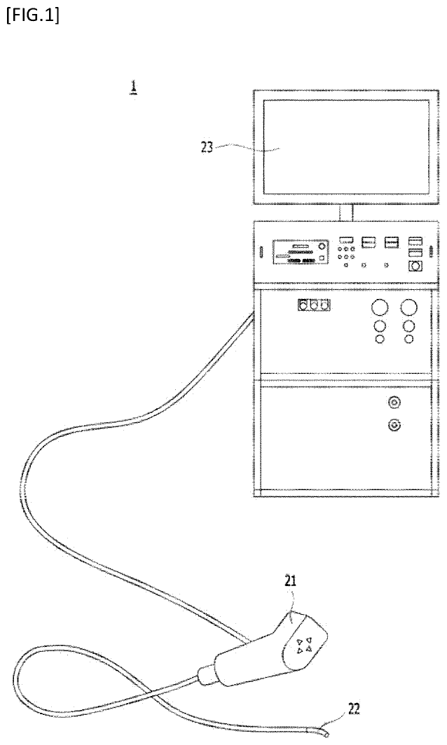

FIG. 1 is a schematic diagram of a lesion diagnostic device according to an exemplary embodiment of the present disclosure.

Referring to FIG. 1, a lesion diagnostic device 10, an endoscopic device 20, and a display device 23 may send and receive data (images, video, and text) and a variety of communication signals over a network. A lesion diagnostic system 1 may include all types of servers, terminals, or devices having data storage and processing functions.

An example of a network for sharing information among the lesion diagnostic device 10, endoscopic device 20, and display device 23 may include, but is not limited to, a 3GPP (3rd Generation Partnership Project) network, an LTE (Long Term Evolution) network, a WI MAX (World Interoperability for Microwave Access) network, the Internet, a LAN (Local Area Network), a Wireless LAN (Wireless Local Area Network), a WAN (Wide Area Network), a PAN (Personal Area Network), a Bluetooth network, a satellite broadcast network, an analog broadcast network, and a DMB (Digital Multimedia Broadcasting) network.

The endoscopic device 20 may be a device used for gastroendoscopic examination. The endoscopic device 20 may include an operation part 21 and a body part 22. The endoscopic device 20 may include a body part 22 to be inserted into the body and an operation part 21 provided on the rear end of the body part 22. An imaging part for imaging the inside of the body, a lighting part for illuminating a target region, a water spray part for washing the inside of the body to facilitate imaging, and a suction part for sucking foreign materials or air from inside the body may be provided on the front end of the body part 22. Channels corresponding to these units (parts) may be provided inside the body part 22. Moreover, a biopsy channel may be provided inside an insertion part, and the endoscopist may take samples of tissue from inside the body by inserting a scalpel through the biopsy channel. The imaging part (i.e., camera) for imaging the inside of the body, provided at the endoscopic device 20, may have a miniature camera. The imaging device may acquire white-light gastroendoscopic images.

The imaging part of the endoscopic device 20 may send and receive acquired gastric lesion images to the lesion diagnostic device 10 over a network. The lesion diagnostic device 10 may generate a control signal for controlling the biopsy unit based on a gastric lesion diagnosis. The biopsy unit may be a unit for taking samples of a tissue from inside the body. The tissue samples taken from inside the body may determine whether the tissue is benign or malignant. Also, cancer tissue can be removed by excision of tissue from inside the body. For example, the lesion diagnostic device 10 may be included in the endoscopic device 20 which acquires gastroendoscopic images and takes samples of tissue from inside the body. In other words, a gastric lesion may be diagnosed and predicted by feeding gastroendoscopic images, acquired in real time from the endoscopic device 20, into an artificial neural network built on training and putting them into at least one of the categories for gastric lesion diagnosis.

According to another exemplary embodiment of the present disclosure, the endoscopic device 20 may be made in capsule form. For example, the endoscopic device 20 may be made in capsule form and inserted into a patient's body to acquire gastroendoscopic images. The capsule endoscopic device 20 also may provide location information which shows where it is located—either in the esophagus, the stomach, the small intestine, or the large intestine. In other words, the capsule endoscopic device 20 may be positioned inside the patient's body and provide real-time images to the lesion diagnostic device 10 over a network. In this case, the capsule endoscopic device 20 may provide information on the locations where the gastroendoscopic images are acquired, as well as the gastroendoscopic images themselves. If the diagnosis by the lesion diagnostic device 10 is classified as at least one of the following categories: advanced gastric cancer, early gastric cancer, high-grade dysplasia, and low-grade dysplasia—that is, non-benign risky tumor, the user (doctor) may identify the location of the lesion and remove it immediately.

According to an exemplary embodiment of the present disclosure, the lesion diagnostic device 10 may perform a gastric lesion diagnosis based on gastric lesion endoscopic images, which are acquired in real time from the endoscopic device 20 and fed into an algorithm generated by training, and the endoscopic device 20 may remove a lesion suspicious for a neoplasm by endoscopic mucosal resection or endoscopic submucosal dissection.

The display device 20 may include, for example, a liquid crystal display LCD, a light-emitting diode (LED) display, an organic light-emitting diode (OLED) display, or a microelectromechanical (MEMS) display. The display device 23 may present the user gastroendoscopic images acquired from the endoscopic device 20 and information on a gastric lesion diagnosis made by the lesion diagnostic device 10. The display device 23 may include a touchscreen—for example, it may receive a touch, gesture, proximity, or hovering input using an electronic pen or a part of the user's body. The display device 23 may output gastroendoscopic images acquired from the endoscopic device 20. Also, the display device 23 may output gastric lesion diagnostic results.

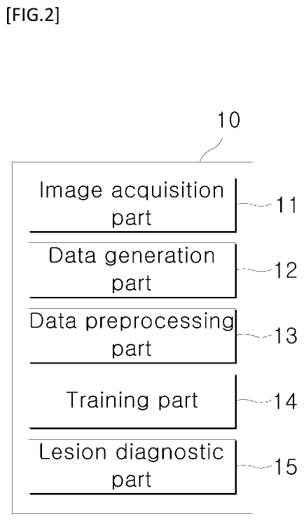

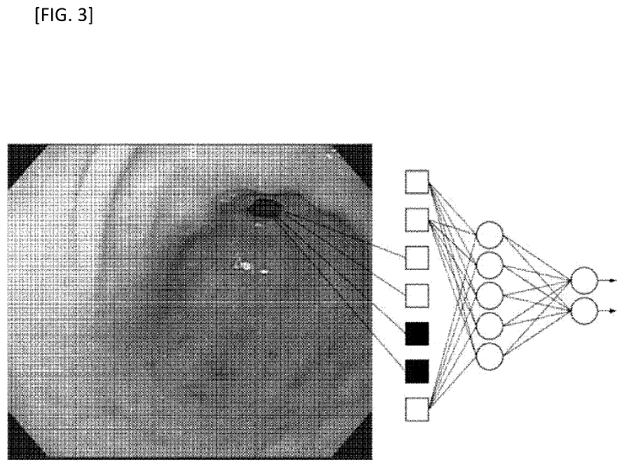

FIG. 2 is a schematic block diagram of a lesion diagnostic device according to an exemplary embodiment of the present disclosure. FIG. 3 is a view illustrating an example of building an artificial neural network in a lesion diagnostic device according to an exemplary embodiment of the present disclosure.

Referring to FIG. 2, the lesion diagnostic device 10 may include an image acquisition part 11, a data generation part 12, a data preprocessing part 13, a training part 14, and a lesion diagnostic part 15. However, the components of the lesion diagnostic device 10 are not limited to those disclosed above. For example, the lesion diagnostic device 10 may further include a database for storing information.

The image acquisition part 11 may acquire a plurality of gastric lesion images. The image acquisition part 11 may receive gastric lesion images from an imaging device provided in the endoscopic device 20. The image acquisition part 11 may acquire gastric lesion images acquired with an endoscopic video imaging device (digital camera) used for gastroendoscopy. The image acquisition part 11 may collect white-light gastroendoscopic images of a pathologically confirmed lesion. Also, the image acquisition part 11 may receive a plurality of gastric lesion images from a plurality of hospitals' image storage devices and database systems. The plurality of hospitals' image storage devices may be devices that store gastric lesion images acquired during gastroendoscopy in multiple hospitals.

Moreover, the image acquisition part 11 may acquire images that are taken by varying either the angle, direction, or distance of a first area in the patient's stomach. The image acquisition part 11 may acquire gastric lesion images in JPEG format. The gastric lesion images may be styled with a 35-degree field of view at 1280×640 pixel resolution. Meanwhile, the image acquisition part 11 may acquire gastric lesion images from which their individual identifier information has been removed. The image acquisition part 11 may acquire gastric lesion images where the lesion is located at the center and where the black frame area has been removed.

On the contrary, if the image acquisition part 11 acquires images of low quality or low resolution, such as out-of-focus images, images including at least one artifact, and low-dynamic-range images, these images may be excluded. In other words, the image acquisition part 11 may exclude images if they are not applicable to a deep learning algorithm.

According to an exemplary embodiment of the present disclosure, the endoscopic device 20 may control the imaging part by using the operation part 21. The operation part 21 may receive an operation input signal from the user in order that the imaging part has a target lesion within its field of view. The operation part 21 may control the position of the imaging part based on an operation input signal inputted from the user. Also, if the field of view of the imaging part covers the target lesion, the operation part 21 may receive an operation input signal for capturing a corresponding image and generate a signal for capturing the corresponding gastric lesion image.

According to another exemplary embodiment of the present disclosure, the endoscopic device 20 may be a device that is made in capsule form. The capsule endoscopic device 20 may be inserted into the body of a patient and remotely operated. Gastric lesion images acquired from the capsule endoscopic device 20 may include all images acquired by video recording, as well as images of a region the user wants to capture. The capsule endoscopic device 20 may include an imaging part and an operation part. The imaging part may be inserted into a human body and controlled inside the human body based on an operation signal from the operation part.

The data generation part 12 may generate a dataset by linking a plurality of gastric lesion images with patient information. The patient information may include the patient's sex, age, height, weight, race, nationality, smoking status, alcohol intake, and family history. Furthermore, the patient information may include clinical information. The clinical information may refer to all data a doctor can use when making a specific diagnosis in a hospital. Particularly, the clinical information may include electronic medical records containing personal information like sex and age, specific medical treatments received, billing information, and orders and prescriptions, which are created throughout a medical procedure. Moreover, the clinical information may include biometric data such as genetic information. The biometric data may include personal health information containing numerical data like heart rate, electrocardiogram, exercise and movement levels, oxygen saturation, blood pressure, weight, and blood sugar level.

The patient information is data that is fed into a fully-connected neural network, along with the output from the convolutional neural network architecture, from the training part 14 to be described later, and further improvements in accuracy can be expected by feeding other information other than gastric lesion images as input into an artificial neural network.

Moreover, the data generation part 12 may generate a training dataset and a validation dataset, for use on a deep learning algorithm. A dataset, when generated, may be classified as a training dataset required for training the artificial neural network or a validation dataset for validating information on the progress of the training of the artificial neural network. For example, the data generation part 12 may classify gastric lesion images acquired by the image acquisition part 11 into images to be randomly used for a training dataset and images used for a validation dataset. Also, the data generation part 12 may use all other images, except for those used for the validation dataset, as the training dataset. The validation dataset may be randomly selected. The percentage of the validation dataset and the percentage of the training dataset may take on preset reference values. The preset reference values may be 10% for the validation dataset and 90% for the training dataset, respectively, but not limited thereto.

The data generation part 12 may generate the training dataset and the validation dataset separately in order to avoid overfitting. For example, neural network architectures may be overfitted to the training dataset due to their learning characteristics. Thus, the data generation part 12 may use the validation dataset to avoid overfitting of the artificial neural network.

The validation dataset may be a dataset that is not redundant with the training dataset. Since validation data is not used for building an artificial neural network, the validation data is the first data that the artificial neural network will encounter during validation. Accordingly, the validation dataset may be suitable for evaluating the performance of the artificial neural network when new images (not used for training) are fed as input.

The preprocessing part 13 may preprocess a dataset in a way that is applicable to a deep learning algorithm. The preprocessing part 13 may preprocess a dataset in order to enhance the recognition performance of the deep learning algorithm and minimize similarities between different patients' images. The deep learning algorithm may be composed of two parts: a convolutional neural network architecture and a fully-connected neural network architecture.

According to an exemplary embodiment of the present disclosure, the preprocessing part 13 may perform a preprocessing process in five phases. First of all, the preprocessing part 13 may perform a cropping phase. In the cropping phase, an unnecessary portion (on a black background) on the edge around a lesion may be cropped from a gastric lesion image acquired by the image acquisition part 11. For example, the preprocessing part 13 may cut the gastric lesion image to an arbitrarily specified pixel size (e.g., 299×299 pixels or 244×244 pixels). In other words, the preprocessing part 13 may cut the gastric lesion image to a size applicable for the deep learning algorithm.

Next, the preprocessing part 13 may perform a parallel shifting phase. The preprocessing part 13 may shift the gastric lesion image in parallel upward, downward, to the left, or to the right. Also, the preprocessing part 13 may perform a flipping phase. For example, the preprocessing part 13 may flip the gastric lesion image vertically. Also, the preprocessing part 13 may flip the gastric lesion image upward or downward and then flip it to the left or right.

Moreover, the preprocessing part 13 may perform a color adjustment phase. For example, in the color adjustment phase, the preprocessing part 13 may perform color adjustment on an image based on colors extracted by computing the mean RGB values across the entire dataset and subtracting them from the image. Also, the preprocessing part 13 may randomly adjust colors in the gastric lesion image.

The preprocessing part 13 may generate a dataset of gastric lesion images applicable to the deep learning algorithm by performing the five phases of the preprocessing process. Also, the preprocessing part 13 may generate a dataset of gastric lesion images applicable to the deep learning algorithm by performing at least one of the five phases of the preprocessing process.

Furthermore, the preprocessing part 13 may perform a resizing phase. The resizing phase may be a phase in which a gastric lesion image is enlarged or reduced to a preset size.

The preprocessing part 13 may include an augmentation part (not shown) for augmenting image data to increase the amount of gastric lesion image data.

According to an exemplary embodiment of the present disclosure, in the case of a deep learning algorithm including a convolutional neural network, the greater the amount of data, the better the performance. However, the amount of gastroendoscopic images from endoscopic examinations is much less than the amount of images from other types of examinations, and therefore the amount of gastric lesion image data collected and detected by the image acquisition part 11 may be very insufficient for use on a convolutional neural network. Thus, the augmentation part (not shown) may perform a data augmentation process based on a training dataset. The augmentation part (not shown) may perform a data augmentation process by applying at least one of the following: rotating, flipping, cropping, and adding noise into gastric lesion images.

The preprocessing part 13 may perform a preprocessing process in a way that corresponds to a preset reference value. The preset reference value may be arbitrarily specified by the user. Also, the preset reference value may be determined by an average value for acquired gastric lesion images. A dataset may be provided to the training part 14 once it has undergone the preprocessing part 13.

The training part 14 may build an artificial neural network by training the artificial neural network by using a preprocessed dataset as input and gastric lesion classification results as output.

According to an exemplary embodiment of the present disclosure, the training part 14 may provide gastric lesion classification results as output by applying a deep learning algorithm consisting of two parts: a convolutional neural network architecture and a fully-connected neural network architecture. The fully-connected neural network is a neural network in which nodes are two-dimensionally interconnected horizontally and longitudinally and there are interconnections between nodes on adjacent layers but not between nodes within the same layer.

The training part 14 may build a training model in which a convolutional neural network is trained by taking a preprocessed training dataset as input and a fully-connected neural network is trained by taking the output of the convolutional neural network as input.

According to an exemplary embodiment of the present disclosure, the convolutional neural network may extract a plurality of specific feature patterns by analyzing gastric lesion images. The extracted specific feature patterns may be used for final classification in the fully-connected neural network.

Convolutional neural networks are a type of neural network mainly used for speech recognition or image recognition. Since the convolutional neural network is constructed to process multidimensional array data, it is specialized for processing a multidimensional array such as a color image array. Accordingly, most techniques using deep learning in image recognition are based on convolutional neural networks.

For example, referring to FIG. 3, the convolutional neural network CNN processes an image by partitioning it into multiple segments, rather than using the whole image as a single piece of data. This can extract local features of the image even if the image is distorted, thereby allowing the convolutional neural network CNN to deliver proper performance.

The convolutional neural network may consist of a plurality of layers. The elements of each layer may include a convolutional layer, an activation function, a max pooling layer, an activation function, and a dropout layer. The convolutional layer serves as a filter called a kernel to locally process the entire image (or a newly generated feature pattern) and extract a new feature pattern of the same size as the image. For a feature pattern, the convolutional layer may correct the values of the feature pattern through the activation function to make it easier to process them. The max pooling layer may take a sample from a gastric lesion image and reduce the size of the image by size adjustment. Although feature patterns are reduced in size as they pass through the convolutional layer and the max pooling layer, the convolutional neural network may extract a plurality of feature patterns by using a plurality of kernels. The dropout layer may involve a method in which, when training the weights of the convolutional neural network, some of the weights are not used deliberately for efficient training. Meanwhile, the dropout layer may not be applied when actual testing is performed through a training model.

A plurality of feature patterns extracted from the convolutional neural network may be delivered to the following phase, i.e., the fully-convolutional neural network, and used for classification. The convolutional neural network may adjust the number of layers. By adjusting the number of layers in the convolutional neural network to fit the amount of training data required for model training, the model can be built with higher stability.

Moreover, the training part 14 may build a diagnostic (training) model in which a convolutional neural network is trained by taking a preprocessed training dataset as input and a fully-connected neural network is trained by taking the output of the convolutional neural network and the patient information as input. In other words, the training part 14 may allow preprocessed image data to preferentially enter the convolutional neural network and allow the output of the convolutional neural network to enter the fully-connected neural network. Also, the training part 14 may allow randomly extracted features to directly enter the fully-connected neural network without passing through the convolutional neural network.

In this case, the patient information may include various information such as the patient's sex, age, height, weight, race, nationality, smoking status, alcohol intake, and family history. Furthermore, the patient information may include clinical information. The clinical information may refer to all data a doctor can use when making a specific diagnosis in a hospital. Particularly, the clinical information may include electronic medical records containing personal information like sex and age, specific medical treatments received, billing information, and orders and prescriptions, which are created throughout a medical procedure. Moreover, the clinical information may include biometric data such as genetic information. The biometric data may include personal health information containing numerical data like heart rate, electrocardiogram, exercise and movement levels, oxygen saturation, blood pressure, weight, and blood sugar levels.

The patient information is data that is fed into a fully-connected neural network, along with the output of the convolutional neural network architecture, from the training part 14, and further improvements in accuracy can be expected by feeding the patient information as input into an artificial neural network, rather than deriving the output by using gastric lesion images alone.

For example, once training is done on clinical information in a training dataset, indicating that the incidence of cancer increases with age, the input of an age 42 or 79, along with image features, may derive gastric lesion classification results showing that older patients with an uncertain lesion that is hard to classify as benign or malignant have a higher probability of cancer.

The training part 14 may perform training by applying training data to a deep learning algorithm architecture (an architecture in which the training data is fed into the fully-connected neural network through the convolutional neural network), calculating the error between the output derived from the training data and the actual output, and giving feedback on the outputs through a backpropagation algorithm to gradually change the weights of the neural network architecture by an amount corresponding to the error. The backpropagation algorithm may adjust the weight between each node and its next node in order to reduce the output error (difference between the actual output and the derived output). The training part 14 may derive a final diagnostic model by training the neural networks on a training dataset and a validation dataset and calculating weight parameters.

The lesion diagnostic part 15 may perform a gastric lesion diagnosis through an artificial neural network after passing a new dataset through a preprocessing process. In other words, the lesion diagnostic part 15 may derive a diagnosis on new data by using the final diagnostic model derived by the training part 14. The new data may include gastric lesion images based on which the user wants to make a diagnosis. The new dataset may be a dataset that is generated by linking gastric lesion images with patient information. The new dataset may be preprocessed such that it becomes applicable to a deep learning algorithm after passing through the preprocessing process of the preprocessing part 13 Afterwards, the preprocessed new dataset may be fed into the training part 14 to make a diagnosis with respect to the gastric lesion images based on training parameters.

According to an exemplary embodiment of the present disclosure, the lesion diagnostic part 15 may classify a gastric lesion diagnosis as at least one of the following categories: advanced gastric cancer, early gastric cancer, high-grade dysplasia, and low-grade dysplasia. Moreover, the lesion diagnostic part 15 may diagnose and classify gastric lesions as cancerous or non-cancerous. Also, the lesion diagnostic part 15 may diagnose and classify gastric lesions into two categories: neoplasm and non-neoplasm. The neoplasm category may include AGC, EGC, HGD, and LGD. The non-neoplasm category may include lesions such as gastritis, benign ulcers, erosions, polyps, or intestinal metaplasia, and epithelial tumor.

The lesion diagnostic device 10 may analyze images acquired by the endoscopic device 20 and automatically classify and diagnose uncertain lesions, in order to reduce side effects of an unnecessary biopsy or endoscopic excision performed to classify and diagnose uncertain lesions, and may allow the doctor to proceed with an endoscopic excision treatment in the case of a neoplasm (dangerous tumor).

According to another exemplary embodiment of the present disclosure, the endoscopic device 20 may include an operation part 21, a body part 22, a controller 23, a lesion location acquisition part 24, and a display 25.

The operation part 21 may be provided on the rear end of the body part 22 and manipulated based on information inputted by the user. The operation part 21 is a part that is gripped by an endoscopist, with which the body part 22 to be inserted into the patient's body. Also, the operation part 21 allows for manipulating the operation of a plurality of units required for an endoscopic procedure the body part 22 contains. The operation part 21 may include a rotary controller. The rotary controller may include a part that functions to generate a control signal and provides rotational force (such as in a motor). The operation part 21 may include buttons for manipulating the imaging part (not shown). The buttons are used to control the position of the imaging part (not shown), by which the user may change the position of the body part 22 upward, downward, to the left, to the right, forward, backward, and so forth.

The body part 22 is a part that is inserted into the patient's body, and may contain a plurality of units. The plurality of units may include at least one of an imaging part (not shown) for imaging the inside of the patient's body, an air supply unit for supplying air into the body, a water supply unit for supplying water into the body, a lighting unit for illuminating the inside of the body, a biopsy unit for sampling a portion of tissue in the body or treating the tissue, and a suction unit for sucking air or foreign materials from inside the body. The biopsy unit may include a variety of medical instruments, such as scalpels, needles, and so on, for sampling a portion of tissue from a living organism, and the scalpels and needles in the biopsy unit may be inserted into the body through a biopsy channel by the endoscopist to sample cells in the body.

The imaging part (not shown) may hold a camera of a size equivalent to the diameter of the body part 22. The imaging part (not shown) may be provided on the front end of the body part 22 and take gastric lesion images and provide the taken gastric lesion images to the lesion diagnostic part 10 and the display 25 over a network.

The controller 23 may generate a control signal for controlling the operation of the body part 22 based on user input information provided from the operation part 21 and the diagnostic results of the lesion diagnostic device 10. Upon receiving an input from the user made by selecting one of the buttons on the operation part 21, the controller 23 may generate a control signal for controlling the operation of the body part 22 to correspond to the selected button. For example, if the user selects the forward button for the body part 22, the controller 23 may generate an operation control signal to enable the body part 22 to move forward inside the patient's body at a constant speed. The body part 22 may move forward inside the patient's body based on a control signal from the controller 23.

Moreover, the controller 23 may generate a control signal for controlling the operation of the imaging part (not shown). The control signal for controlling the operation of the imaging part (not shown) may be a signal for allowing the imaging part (not shown) positioned in a lesion area to capture a gastric lesion image. In other words, if the user wants the imaging part (not shown) positioned in a specific lesion area to acquire an image based on an input from the operation part 21, they may click on a capture button. The controller 23 may generate a control signal to allow the imaging part (not shown) to acquire an image in the lesion area based on input information provided from the operation part 21. The controller 23 may generate a control signal for acquiring a specific lesion gastric image from the video the imaging part (not shown) is recording.

Additionally, the controller 23 may generate a control signal for controlling the operation of the biopsy unit for sampling a portion of tissue in the patient's body based on the diagnostic results of the lesion diagnostic device 10. If the diagnosis by the lesion diagnostic device 10 is classified as at least one of the following categories: advanced gastric cancer, early gastric cancer, high-grade dysplasia, and low-grade dysplasia, the controller 23 may generate a control signal for controlling the operation of the biopsy unit to perform an excision. The biopsy unit may include a variety of medical instruments, such as scalpels, needles, and so on, for sampling a portion of tissue from a living organism, and the scalpels and needles in the biopsy unit may be inserted into the body through a biopsy channel by the endoscopist to sample cells in the body. Also, the controller 23 may generate a control signal for controlling the operation of the biopsy unit based on a user input signal provided from the operation part 21. The user may perform the operation of sampling, excising, or removing cells inside the body by using the operation part 21.

According to an exemplary embodiment of the present disclosure, the lesion location acquisition part 24 may generate gastric lesion information by linking the gastric lesion images provided from the imaging part (not shown) with location information. The location information may be information on the current location of the body part 22 inside the body. In other words, if the body part 22 is positioned at a first point on the stomach of the patient's body and a gastric lesion image is acquired from the first point, the lesion location acquisition part 24 may generate gastric lesion information by linking this gastric lesion image with the location information.

The lesion location acquisition part 24 may provide the user (doctor) with the gastric lesion information generated by linking the acquired lesion gastric lesion images with the location information. By providing the user with the diagnostic results of the lesion diagnostic part 10 and the gastric lesion information of the gastric lesion location acquisition part 24 through the display 25, the risk of excision somewhere else other than the target lesion may be avoided when performing an excision treatment or surgery on the target lesion.

Moreover, if the biopsy unit is not positioned in the target lesion based on the location information provided from the lesion location acquisition part 24, the controller 23 may generate a control signal for controlling the position of the biopsy unit.

Since the lesion diagnostic device 10 generates a control signal for controlling the biopsy unit and samples or removes cells from inside the body, tissue examinations can be made much faster. Besides, the patient can be treated quickly since cells diagnosed as cancer can be removed immediately during an endoscopic diagnosis procedure.

Hereinafter, the operation flow of the present disclosure will be discussed briefly based on what has been described in detail above.

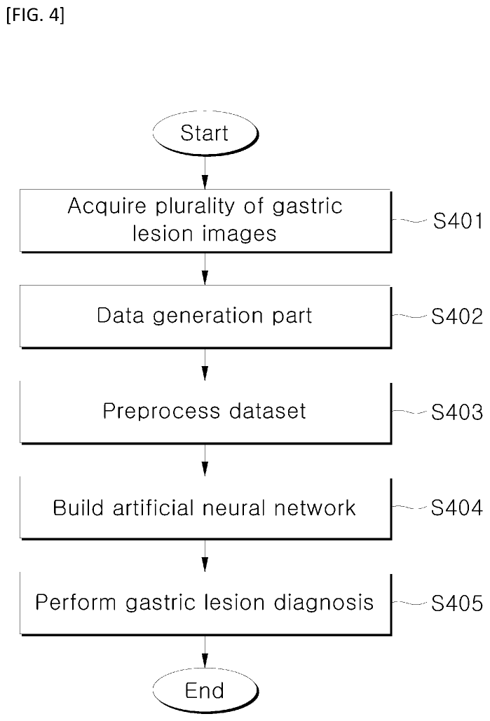

FIG. 4 is an operation flow chart of a method for diagnosing a gastric lesion from endoscopic images according to an exemplary embodiment of the present disclosure.

The method for diagnosing a gastric lesion from endoscopic images, shown in FIG. 4, may be performed by the above-described lesion diagnostic device 10. Thus, the description of the lesion diagnostic device 10 may be omitted since it may apply equally to the method for diagnosing a gastric lesion from an endoscopic.

In the step S401, the lesion diagnostic device 10 may acquire a plurality of gastric lesion images. The lesion diagnostic device 10 may receive the acquired gastric lesion images from the imaging device with which the endoscopic device 20 is equipped. The gastric lesion images may be white-light images.

In the step S402, the lesion diagnostic device 10 may generate a dataset by linking a plurality of gastric lesion images with patient information. When generating a dataset, the lesion diagnostic device 10 may classify the dataset as a training dataset required for training the artificial neural network or a validation dataset for validating information on the progress of the training of the artificial neural network. In this case, the validation dataset may be a dataset that is not redundant with the training dataset. The validation dataset may be used for evaluating the performance of the artificial neural network when a new dataset is fed as input into the artificial neural network after passing through the preprocessing process.

In the step S403, the lesion diagnostic device 10 may preprocess a dataset in a way that is applicable to a deep learning algorithm. The lesion diagnostic device 10 may perform a cropping process in which a peripheral area of a gastric lesion image included in the dataset is cropped around the gastric lesion to a size applicable for the deep learning algorithm in such a way that the gastric lesion is not included in the image. Also, the lesion diagnostic device 10 may shift the gastric lesion image in parallel upward, downward, to the left, or to the right. Also, the lesion diagnostic device 10 may flip the gastric lesion image. Also, the lesion diagnostic device 10 may adjust colors in the gastric lesion image. The lesion diagnostic device 10 may preprocess the gastric lesion image in a way that is applicable to the deep learning algorithm.

Moreover, the lesion diagnostic device 10 may augment image data to increase the amount of gastric lesion image data. The lesion diagnostic device 10 may augment gastric lesion image data by applying at least one of the following: rotating, flipping, cropping, and adding noise into the gastric lesion image data.

In the step S404, the lesion diagnostic device 10 may build an artificial neural network by training the artificial neural network by using a preprocessed dataset as input and gastric lesion classification results as output. The lesion diagnostic device 10 may build a training model in which a convolutional neural network and a fully-connected neural network are trained by using the preprocessed dataset as input and the gastric lesion classification results of the convolutional neural network as output.

In addition, the lesion diagnostic device 10 may build a training model in which a convolutional neural network is trained by taking a preprocessed dataset as input and a fully-connected neural network is trained by taking the output of the convolutional neural network and the patient information as input. The convolutional neural network may output a plurality of feature patterns from a plurality of gastric lesion images, and the plurality of feature patterns may be finally classified by the fully-connected neural network.

In the step S405, the lesion diagnostic device 10 may perform a gastric lesion diagnosis through the artificial neural network after passing a new dataset through the preprocessing process. The lesion diagnostic device 10 may classify a gastric lesion diagnosis as at least one of the following categories: advanced gastric cancer, early gastric cancer, high-grade dysplasia, and low-grade dysplasia.

In the above description, the steps S401 to S405 may be further subdivided into a greater number of steps or combined into a smaller number of steps in some examples of implementation of the present disclosure. Moreover, some of the steps may be omitted if necessary, or the sequence of the steps may be changed.

A method for diagnosing a gastric lesion from endoscopic images according to an exemplary embodiment of the present disclosure may be realized in the form of program instructions which can be implemented through various computer components, and may be recorded in a computer-readable storage medium. The computer-readable storage medium may include program instructions, a data file, a data structure, and the like either alone or in combination thereof. The program instructions recorded in the computer-readable storage medium may be any program instructions particularly designed and structured for the present disclosure or known to those skilled in the field of computer software. Examples of the computer-readable storage medium include magnetic recording media, such as hard disks, floppy disks and magnetic tapes, optical data storage media, such as CD-ROMs and DVD-ROMs, magneto-optical media such as floptical disks, and hardware devices, such as read-only memories (ROMs), random-access memories (RAMs), and flash memories, which are particularly structured to store and implement the program instructions. Examples of the program instructions include not only assembly language code formatted by a compiler but also high-level language code which can be implemented by a computer using an interpreter. The hardware device described above may be configured to operate as one or more software modules to perform operations of the present disclosure, and vice versa.

In addition, the above-described method for diagnosing a gastric lesion from endoscopic images also may be implemented in the form of a computer-executable computer program or application stored in a recording medium.

Although some embodiments have been described herein, it should be understood that these embodiments are provided for illustration and that various modifications, changes, alterations, and equivalent embodiments can be made by those skilled in the art without departing from the spirit and scope of the present disclosure. Therefore, the embodiments are not to be construed in any way as limiting the present disclosure. For example, each component described as a single type may be implemented in a distributed manner, and, similarly, components described as distributed may be implemented in a combined form.

The scope of the present application should be defined by the appended claims and equivalents thereof rather than by the detailed description, and all changes or modifications derived from the spirit and scope of the claims and equivalents thereof should be construed as within the scope of the present disclosure.

1. A method for diagnosing a gastric lesion from endoscopic images, the method comprising:
acquiring a plurality of gastric lesion images;
generating a dataset by linking the plurality of gastric lesion images with patient information;
preprocessing the dataset in a way that is applicable to a deep learning algorithm; and
building an artificial neural network by training the artificial neural network by using the preprocessed dataset as input and gastric lesion classification results as output.

2. The method of claim 1, further comprising performing a gastric lesion diagnosis through the artificial neural network after passing a new dataset through the preprocessing process.

3. The method of claim 1, wherein the generating of a dataset comprises classifying the dataset as a training dataset required for training the artificial neural network or a validation dataset for validating information on the progress of the training of the artificial neural network.

4. The method of claim 3, wherein the validation dataset is a dataset that is not redundant with the training dataset.

5. The method of claim 3, wherein the validation dataset is used for evaluating the performance of the artificial neural network when a new dataset is fed as input into the artificial neural network after passing through the preprocessing process.

6. The method of claim 1, wherein the acquisition of images comprises receiving gastric lesion images acquired by an imaging device with which an endoscopic device is equipped.

7. The method of claim 1, wherein the preprocessing comprises:
cropping a peripheral area of a gastric lesion image included in the dataset around the gastric lesion to a size applicable for the deep learning algorithm in such a way that the gastric lesion is not included in the image;
shifting the gastric lesion image in parallel upward, downward, to the left, or to the right;
rotating the gastric lesion image;
flipping the gastric lesion image; and
adjusting colors in the gastric lesion image,
wherein the gastric lesion image is preprocessed in a way that is applicable to the deep learning algorithm by performing at least one of the preprocessing phases.

8. The method of claim 7, wherein the preprocessing comprises augmenting image data to increase the amount of gastric lesion image data,
wherein the augmenting of image data comprises augmenting the gastric lesion image data by applying at least one of the following: rotating, flipping, cropping, and adding noise into the gastric lesion image data.

9. The method of claim 1, wherein the building of a training model comprises building a training model in which a convolutional neural network and a fully-connected neural network are trained by using the preprocessed dataset as input and the gastric lesion classification results as output.

10. The method of claim 9, wherein the preprocessed dataset is fed as input into the convolutional neural network, and the output of the convolutional neural network and the patient information are fed as input into the fully-connected neural network.

11. The method of claim 10, wherein the convolutional neural network produces a plurality of feature patterns from the plurality of gastric lesion images,
wherein the plurality of feature patterns are finally classified by the fully-connected neural network.

12. The method of claim 9, wherein the building of an artificial neural network comprises performing training by applying training data to a deep learning algorithm architecture including a convolutional neural network and a fully-connected neural network, calculating the error between the output derived from the training data and the actual output, and giving feedback on the outputs through a backpropagation algorithm to gradually change the weights of the artificial neural network architecture by an amount corresponding to the error.

13. The method of claim 2, wherein the performing of a gastric lesion diagnosis comprises classifying the gastric lesion diagnosis as at least one of the following categories: advanced gastric cancer, early gastric cancer, high-grade dysplasia, and low-grade dysplasia.

14. A device for diagnosing a gastric lesion from endoscopic images, the device comprising:
an image acquisition part for acquiring a plurality of gastric lesion images;
a data generation part for generating a dataset by linking the plurality of gastric lesion images with patient information;
a data preprocessing part for preprocessing the dataset in a way that is applicable to a deep learning algorithm; and
a training part for building an artificial neural network by training the artificial neural network by using the preprocessed dataset as input and gastric lesion classification results as output.

15. The device of claim 14, further comprising a lesion diagnostic device for performing a gastric lesion diagnosis through the artificial neural network after passing a new dataset through the preprocessing process.

16. A computer-readable recording medium storing a program for executing the method of claim 1 on a computer.
